(19) United States
(12) Patent Application Publication
(10) Pub. No.: US 2021/0348052 A1
(43) Pub. Date: Nov. 11, 2021

(54) LIGHT-EMITTING PARTICLE

(71) Applicant: Sumitomo Chemical Company Limited, Tokyo (JP)

(72) Inventors: Jonathan Behrendt, Bedford (GB); Nazrul Islam, Cambourne (GB); Kira Kamtekar, Godmanchester (GB)

(73) Assignee: Sumitomo Chemical Company Limited, Tokyo (JP)

(21) Appl. No.: 17/278,241

(22) PCT Filed: Sep. 13, 2019
(86) PCT No.: PCT/EP2019/074502

§ 371 Date: Mar. 19, 2021

(30) Foreign Application Priority Data
Sep. 20, 2018 (GB) 1815338.7
Jul. 3, 2019 (GB) 1909598.3

(51) Int. Cl. C09K 11/06; C09K 11/02; G01N 33/58

(52) U.S. Cl. CPC C09K 11/06; C09K 11/02; G01N 33/582; C09K 2211/1458; C09K 2211/1425; C09K 2211/1483; C09K 2211/1416

(57) ABSTRACT

A particle having an inorganic matrix material and a light-emitting polymer wherein the light-emitting polymer has a light-emitting group and a host repeat unit, wherein a bandgap of the host repeat unit is greater than that of the light-emitting group, wherein the light-emitting group makes up no more than 10 mol % of the groups of the light-emitting polymer and wherein the polymer has a solubility in water or a Ci-s alcohol at 20° C. of at least 0.1 mg/mL.

### BACKGROUND

Embodiments of the present disclosure relate to light-emitting particles, in particular light-emitting nanoparticles, and the use thereof as a luminescent marker. Embodiments of the present disclosure further relate to methods of preparing said light-emitting particles.

### BACKGROUND

Nanoparticles of silica and a light-emitting material have been disclosed as labelling or detection reagents.

WO 2018/060722 discloses composite particles comprising a mixture of silica and a light-emitting polymer having polar groups.

Nanoscale Res. Lett., 2011, vol. 6, p 328 discloses entrapment of a small molecule in a silica matrix.

Langmuir, 1992, vol. 8, pp 2921-2931 discloses coupling of a dye to a silane coupling agent which is then incorporated into a silica sphere.

J. Mater. Chem., 2013, vol. 1, pp 3297-3304, Behrendt et al. describes silica-LEP nanoparticles where the LEP is covalently bound to the silica. The light emitting polymer has alkoxysilane groups pendant from the polymer backbone which react with the silica monomer during formation of the nanoparticles.

Nanoscale, 2013, vol. 5, pp 8593-8601, Geng et al. describes silica-conjugated polymer (CP) nanoparticles wherein the LEP has pendant non-polar alkyl side chains and where the nanoparticles have a “SiO₂@CP@SiO₂” structure.

Chem. Mater., 2014, vol. 26, pp 1874-1880, Geng et al. discloses poly(9,9-dihexylfluorene-alt-2,1,3-benzothiadiazole) (PFBT) loaded nanoparticles.

### SUMMARY

The present inventors have found that upon exciting a light-emitting polymer, e.g. by photoexcitation, efficiency may be particularly high if the light-emitting polymer is constrained within particles and if the light-emitting units of the light-emitting polymer are provided within a certain molar range.

Accordingly, in some embodiments there is provided a particle having an inorganic matrix material and a light-emitting polymer wherein the light-emitting polymer has a light-emitting group and a host repeat unit wherein a bandgap of the host repeat unit is greater than that of the light-emitting group and wherein the light-emitting group makes up no more than 10 mol % of the groups of the light-emitting polymer.

In some embodiments, the light-emitting group is a repeat unit in the polymer backbone, or a substituent of a repeat unit in the polymer backbone.

In some embodiments, the light-emitting polymer is a conjugated polymer.

In some embodiments, the host repeat unit of a conjugated polymer is an arylene repeat unit which may be unsubstituted or substituted with one or more substituents. In some embodiments, the host arylene repeat unit has no more than 3 fused aromatic rings.

In some embodiments the light-emitting group is a light-emitting repeat unit in a backbone of the polymer and the light-emitting group is unsubstituted or substituted with one or more substituents.

In some embodiments, the light-emitting repeat unit is selected from heteroarylene repeat units and arylamine repeat units which may be unsubstituted or substituted with one or more substituents; and metal complexes. In some embodiments, the light-emitting repeat unit is an iridium or platinum metal complex.

In some embodiments, the host repeat unit is a fluorene repeat unit which may be unsubstituted or substituted with one or more substituents.

In some embodiments, the light-emitting group is a substituent of a repeat unit of the light-emitting polymer.

In some embodiments, the light-emitting group makes up no more than 5 mol % of the polymer.

In some embodiments, an absorption peak wavelength of an absorption spectrum of the light-emitting polymer is at least 100 nm shorter than an emission peak wavelength of an emission spectrum of the light-emitting polymer.

In some embodiments, the particle has a biomolecule binding group configured to bind to a target biomolecule.

In some embodiments, the inorganic matrix includes, or is, silica.

In some embodiments there is provided a colloidal suspension comprising particles as described herein. The liquid may be a protic liquid. The protic liquid may have one or more salts dissolved therein.

In some embodiments there is provided a process for preparing a particle as described herein by polymerisation of a silica monomer in the presence of the light-emitting polymer.

In some embodiments there is provided a method of marking a biomolecule, the method comprising the step of binding the biomolecule to a particle as described herein.

In some embodiments there is provided a light-emitting polymer comprising a light-emitting group and a host repeat unit, wherein a bandgap of the host repeat unit is greater than that of the light-emitting group, wherein the light-emitting group makes up no more than 10 mol % of the light-emitting polymer and wherein the polymer has a solubility in water or a C₁₋₈ alcohol at 20° C. of at least 0.1 mg/mL.

In some embodiments there is provided a light-emitting polymer comprising a light-emitting group and a host repeat unit substituted with an ionic group, wherein a bandgap of the host repeat unit is greater than that of a light-emitting group and wherein the light-emitting group makes up no more than 10 mol % of the light-emitting polymer.

The light-emitting polymer may have a solubility in water or a C₁₋₈ alcohol at 20° C. of at least 0.5 mg/mL or at least 0.7 mg/ml.

In some embodiments the polymer comprises a light-emitting repeat unit comprising the light-emitting group. The polymer may comprise at least one host repeat unit. Each host repeat unit may be substituted with at least one polar group. Preferably, each host repeat unit may be substituted with at least one ionic group. The host repeat unit may be an arylene repeat unit which is substituted with the ionic group and optionally a non-ionic group. The ionic group may be a COO— group. The non-ionic group may or may not be polar.

The light-emitting polymer may be a conjugated polymer. In some embodiments, the light-emitting group is a light-emitting repeat unit in a backbone of the polymer and the light-emitting group is unsubstituted or substituted with one or more substituents.

In some embodiments, the light-emitting repeat unit is selected from heteroarylene repeat units and arylamine repeat units which may be unsubstituted or substituted with one or more substituents.

The backbone of the light-emitting polymer may comprise repeat units of formula (I):

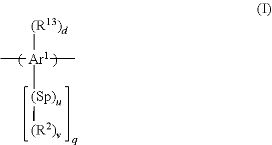

wherein Ar¹ is an arylene group; Sp in each occurrence is independently a spacer group; u is 0 or 1; R² independently in each occurrence is an ionic group; n is 1 if u is 0 and v is at least 1 if u is 1; q is at least 1; R¹³ is a non-ionic group; and d is 0 or a positive integer.

In some embodiments, the repeat unit of formula (I) is a repeat unit of formula (IVa):

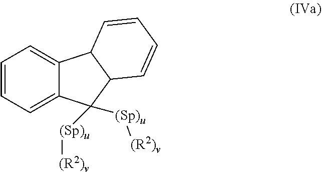

wherein R¹³, Sp, R², u and v are independently in each occurrence as defined previously.

The light-emitting group may be a substituent of a repeat unit of the light-emitting polymer.

In some embodiments, the light-emitting repeat group makes up no more than 5 mol % of the polymer.

In some embodiments, the absorption peak wavelength of an absorption spectrum of the light-emitting polymer is at least 100 nm shorter than an emission peak wavelength of an emission spectrum of the light-emitting polymer.

In some embodiments, there is provided a solution comprising the light-emitting polymer. The solution may be a water, C₁₋₈ alcohol or C₁₋₄ alcohol solution. The polymer in solution may have a solubility of at least about 0.1, 0.5 or 0.7 mg/mL at 20° C.

### DESCRIPTION OF THE DRAWINGS

The disclosed technology and accompanying figures describe some implementations of the disclosed technology.

FIG. 1A shows absorption spectra a nanoparticle containing a green light-emitting polymer according to an embodiment of the present disclosure and for the green light-emitting polymer of the nanoparticle in solution and in a film;

FIG. 1B shows photoluminescence spectra for the solution, film and nanoparticle of FIG. 1A;

FIG. 2A shows absorption spectra for a nanoparticle containing a red light-emitting polymer according to an embodiment of the present disclosure and for the red light-emitting polymer of the nanoparticle in solution and in a film;

FIG. 2B shows photoluminescence spectra for the solution, film and nanoparticle of FIG. 2A;

FIG. 3A shows absorption spectra for a comparative nanoparticle containing a green light-emitting polymer and for the green light-emitting polymer of the comparative nanoparticle in solution and in a film; and

FIG. 3B shows photoluminescence spectra for the solution, film and nanoparticle of FIG. 3A;

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to,” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A particle according to some embodiments of the present disclosure comprises an inorganic matrix and a light-emitting polymer. In some embodiments, the light-emitting polymer is the only light-emitting material of the particle. Optionally, the light-emitting polymer consists of the inorganic matrix and the light-emitting polymer.

In some embodiments, the particle further comprises at least one further light-emitting material. The at least one further light-emitting material may be capable of receiving excitation energy from the light-emitting polymer. The at least one further light-emitting material may be a non-polymeric light-emitting material, e.g. having a molecular weight of 500 Daltons or less. The light-emitting material and the at least one further light-emitting material may form a tandem dye.

In some embodiments, the particles have a number average diameter of no more than 5000 nm, more preferably no more than 2500 nm, 1000 nm, 900 nm, 800 nm, 700 nm, 600 mn, 500 nm or 400 nm as measured by a Malvern. Zetasizer Nano ZS. Preferably the particles comprises particles with a number average diameter of between 5-5000 nm, optionally 5-1000 nm, preferably 5-600 mn, more preferably between 5-500 nm, more preferably between 5-400 nm and most preferably between 5-100 nm as measured by a Malvern Zetasizer Nano ZS.

### Matrix

In some embodiments the inorganic matrix comprises or consists of a matrix material selected from an oxide, optionally silica, alumina or titanium dioxide.

Optionally, the light-emitting polymer is not covalently bound to the matrix material. According to these embodiments, there is no need for the matrix material or the light-emitting polymer to be substituted with reactive groups for forming such covalent bonds, e.g. during formation of the particles.

The matrix may comprise a polymer, for example silica, and chains of the light-emitting polymer may be entangled with, but not covalently bound to, polymer chains of the matrix.

Preferably, at least 50 wt % of the total weight of the particle consists of matrix material. Preferably at least 60, 70, 80, 90, 95, 98, 99, 99.5, 99.9 wt % of the total weight of the particle consists of matrix material.

In some embodiments, the particle comprises the light-emitting polymer homogeneously distributed through the matrix.

In some embodiments, the particle comprises a core comprising or consisting of the light-emitting polymer and a shell comprising or consisting of the matrix.

In some embodiments, a silica matrix as described herein may be formed by polymerisation of a silica monomer in the presence of the light-emitting polymer.

In some embodiments, the polymerisation comprises treating a solution comprising silica monomer and the light-emitting polymer, or by adding a solution of silica monomer to a solution of the light-emitting polymer and a base. Optionally, the solvent of the solutions are water, one or more C₁₋₁₀ alcohols or a combination thereof.

In some embodiments, the process comprises polymerising silica monomer in a solution of the matrix monomer and the light-emitting polymer under acidic conditions.

Polymerising a matrix monomer in the presence of a light-emitting polymer may result in one or more chains of the polymer encapsulated within the particle and/or one or more chains of the polymer extending through a particle.

The particles may be formed in a one-step polymerisation process.

Optionally, the silica monomer is an alkoxysilane, preferably a trialkoxy or tetra-alkoxysilane, optionally a C₁₋₁₂ trialkoxy or tetra-alkoxysilane, for example tetraethyl orthosilicate. The silica monomer may be substituted only with alkoxy groups or may be substituted with one or more groups.

Formation of the particles may be as described in WO 2018/060722, the contents of which are incorporated herein by reference.

In some embodiments, a biomolecule binding group is bound to a surface of the particle. The biomolecule binding group may be bound directly to the surface of the particle group or bound through a surface binding group. The surface binding group may comprise polar groups. Optionally, the surface binding group comprises a polyether chain. By “polyether chain” as used herein is meant a chain having two or more ether oxygen atoms.

The biomolecule binding group may be configured to bind to a target biomolecule. Target biomolecules include without limitation DNA, RNA, peptides, carbohydrates, antibodies, antigens, enzymes, proteins and hormones. It will be understood that the biomolecule binding group may be selected according to the target biomolecule.

Silica at the surface of the particles may be reacted to form a group at the surface capable of binding to a biomolecule binding group. Optionally, silica at the surface is reacted with a siloxane.

### Light-Emitting Polymer

The light-emitting polymer comprises a light-emitting group and a host repeat unit.

The polymer has a solubility in water or a C₁₋₈ alcohol at 20° C. of at least 0.1 mg/ml, optionally at least 0.2, 0.3 or 0.5 mg/ml. Preferably, the polymer has a solubility in water or a C₁₋₈ alcohol at 20° C. of at least 0.5 mg/ml or at least 0.7 mg/ml.

The polymer has a solubility in a C₁₋₄ alcohol, preferably methanol, at 20° C. of at least 0.1 mg/ml, optionally at least 0.2, 0.3 or 0.5 mg/ml. Preferably, the polymer has a solubility in a C₁₋₄ alcohol, preferably methanol, at 20° C. of at least 0.5 mg/ml, or at least 0.7 mg/ml.

A light-emitting repeat unit and/or a light-emitting end group of the light-emitting polymer may comprise the light-emitting group.

The host repeat unit may be substituted with an ionic group. In other words, the host repeat unit may be substituted with one or more ionic groups. In some embodiments, at least about 90%, 91%, 92%, 93%, 94%, 95%, 96%, 97%, 98%, 99% of the host repeat units are substituted with one or more ionic groups. In a preferred embodiment, all (e.g. 100%) of the host repeat units are substituted with one or more ionic groups.

Optionally, the repeat units of the light-emitting polymer consist of the host repeat units in the case where the light-emitting group is an end group of the polymer. Optionally, the light-emitting repeat units of the light-emitting polymer consist of the host repeat units and light-emitting repeat units.

Optionally, the light-emitting polymer comprises at least 50 mol % of the host repeat unit, optionally at least 60 mol % or at least 70 mol %. Preferably, the light-emitting polymer comprises at least 85% or at least 90% of the host repeat unit.

No more than 10 mol %, optionally no more than 5 mol %, of the repeat units of the light-emitting polymer are light-emitting repeat units. Optionally, at least 0.1 mol % of the repeat units of the light-emitting polymer are light-emitting repeat units.

Optionally, the light-emitting polymer comprises at least 90% of the host repeat units and at least 1 mol %, 2 mol %, 3 mol %, 4 mol %, 5 mol %, 6 mol %, 7 mol %, 8 mol %, 9 mol % or 10 mol % of the light-emitting repeat units.

Optionally, end groups of the light-emitting polymer, and therefore any light-emitting end groups of the polymer, are present in an amount of no more than 5 mol %, 4 mot %, 3 mol %, 2 mol %, 1 mol %, or 0.5 mol % compared to the number of repeat units of the polymer.

The quantity of a light-emitting group in a light-emitting polymer may be determined by NMR techniques including, without limitation ¹H NMR and/or ¹³C NMR.

In the case where the light-emitting group is comprised in a light-emitting repeat unit of the light-emitting polymer, the percentage of light-emitting repeat units comprising the light-emitting group may be taken to be the percentage of monomers containing the light-emitting group in the polymerisation mixture used to form the polymer.

The repeat units of the light-emitting polymer other than light-emitting repeat units may be host repeat units having a wider HOMO-LUMO bandgap than the light-emitting group.

The polymer may contain only one host repeat unit. The polymer may contain two or more different host repeat units.

The bandgap of a host repeat unit may be the bandgap of a monomer for forming the host repeat unit. The bandgap of a light-emitting group may be the bandgap of a monomer or end-forming group for forming, respectively, a light-emitting repeat unit or an end group comprising the light-emitting group. Preferably, the bandgap of the monomer for forming the host repeat unit, light-emitting repeat unit or an end group comprising the light-emitting group is greater than the bandgap of the host repeat unit, light-emitting repeat unit and end group comprising the light-emitting group, respectively.

HOMO and LUMO levels as described herein may be determined by square wave voltammetry.

Square wave voltammetry measurements may be carried out using a CHI660D Electrochemical workstation with software (IJ Cambria Scientific Ltd), a CHI 104 3 mm Glassy Carbon Disk Working Electrode (IJ Cambria Scientific Ltd), a platinum wire auxiliary electrode and an Ag/AgCl reference electrode (Harvard Apparatus Ltd). The sample may be measured as a dilute solution in a suitable solvent, e.g. toluene. The measurement cell contains the electrolyte, glassy carbon working electrode, platinum counter electrode, and Ag/AgCl reference glass electrode. Ferrocene may be added into the cell at the end of the experiment as reference material (LUMO (ferrocene)=−4.8 eV).

Preferably, an absorption peak wavelength of an absorption spectrum of the light-emitting polymer is at least 100 nm shorter than an emission peak wavelength of an emission spectrum of the light-emitting polymer.

The host repeat units may be hole-transporting repeat units or electron transporting repeat units.

In use, some light emission from host repeat units may be observed. Optionally, little or no emission from host repeat units is observed.

A light-emitting repeat unit may comprise a light-emitting group in the backbone of the polymer or pendant from the polymer backbone.

The light-emitting polymer may have a linear, branched or crosslinked backbone.

The light-emitting polymer may emit fluorescent light, phosphorescent light or a combination thereof.

The light-emitting polymer may be a conjugated polymer. By “conjugated polymer” is meant a polymer comprising adjacent repeat units in the polymer backbone which are directly conjugated to one another. Conjugated light-emitting polymers include, without limitation, polymers comprising one or more of arylene, heteroarylene, arylamine and vinylene, e.g. arylene vinylene, groups conjugated to one another along the polymer backbone.

Conjugation of host repeat units and light-emitting repeat units in the backbone of a conjugated polymer may change the HOMO and LUMO levels of the polymer as compared to the individual repeat units. It will be understood that the bandgap of repeat units of a conjugated polymer as described herein is as measured for the monomers of the repeat units or is less than the monomers of the repeat units.

The light-emitting polymer may be a non-conjugated polymer. The non-conjugated polymer may comprise ethylene repeating units in the polymer backbone, e.g. a polyacrylate or polymethacrylate, wherein some repeat units are light-emitting repeat units substituted with a light-emitting groups. At least some repeat units of a non-conjugated polymer, other than the light-emitting repeat units, may be host repeat units substituted with a host group capable of absorbing excitation energy and transferring the energy to the light-emitting repeat unit. Exemplary host groups include hole-transporting and electron transporting groups, for example carbazole.

The light-emitting repeat unit may be selected according to the desired colour of emission of the light-emitting polymer.

The wavelength or wavelengths at which the light-emitting polymer absorbs light may be different to the wavelength or wavelengths at which the light-emitting polymer emits light. The light-emitting polymers described herein may have a photoluminescence spectrum with one or more than one emission peak. Preferably, the light-emitting polymer exhibits a Stokes shift of between about 25 and about 400 nm, between about 50 and about 300 nm, or between about 100 and about 300 nm.

In some embodiments, the peaks are separated by greater than about 25 nm, about 50 nm, about 100 nm, about 150 nm, about 200 nm or about 250 nm. Preferably, the peaks are separated by more than about 50 nm or about 100 nm.

The full width half maximum (FWHM) of the emission peaks may be different. In some embodiments, one of the emission peaks may have a broader FWHM than one or more of the other emission peaks.

At least one of the peaks of the light-emitting polymers may have a relatively narrow FWHM. Where a polymer has photoluminescence spectrum having more than one peak, a relatively small FWHM may improve the distinction between the different peaks.

In some embodiments, the FWHM of at least one of the emission peaks is greater than about 25 nm, greater than about 50 nm, greater than about 75 nm, greater than about 100 nm, greater than about 125 nm or greater than about 150 nm. The FWHM of at least one of the emission peaks may be between about 25 nm and about 300 nm, between about 50 nm and about 250 nm, between about 75 and about 225 nm, between about 100 and about 200 nm. The maximum FWHM for at least one of the emission peaks may be 300 nm, about 250 nm, about 200 nm or about 150 nm.

The emission peak having the highest intensity may have a FHWM that is greater than the FHWM of the less-intense emission peaks.

A blue light-emitting polymer as described herein may have a photoluminescence spectrum with a peak of no more than 500 nm, preferably in the range of 400-500 nm, optionally 400-490 nm. In some embodiments, the light-emitting polymer may have a photoluminescence spectrum with a peak of between about 400 nm and about 470 nm.

A green light-emitting polymer as described herein may have a photoluminescence spectrum with a peak of more than 500 nm up to 580 nm, optionally more than 500 nm up to 540 nm.

A red light-emitting polymer as described herein may have a photoluminescence spectrum with a peak of no more than more than 580 nm up to 630 nm, optionally 585 nm up to 625 nm.

A near-infrared light-emitting polymer as described herein may have a photoluminescence spectrum with a peak of between 650 nm up to 1200 nm, optionally 700 nm up to 1200 nm.

The photoluminescence spectrum of light-emitting materials as described herein may be as measured in solution using an Ocean Optics 2000+ spectrometer.

In some embodiments, the host repeat units of a conjugated polymer comprise or consist of phenylene repeat units or fused aromatic repeat units having no more than 3 fused aromatic rings, for example fluorene, indenofluorene, benzofluorene, dihydrophenanthrene, phenanthrene, naphthalene and anthracene repeat units.

In some embodiments, the light-emitting repeat units of a conjugated polymer are selected from heteroarylene repeat units; aromatic repeat units having more than 3 fused aromatic rings, e.g. perylene repeat units; thiophene repeat units; benzothiadiazole repeat units; amine repeat units; and arylamine repeat units.

The present inventors have found that the solubility of the light-emitting polymer may be adjusted by selection of one or both of substituents. Light-emitting polymers which are soluble in polar solvents as described herein may be used in, e.g.:
polymerisation of a silane in the presence of the light-emitting polymer in a polar solvent, such as by the Stöber process, to form particles containing silica and the light-emitting polymer; and/or
an assay in a polar solvent using the light-emitting polymer as a fluorescent tag.

One or more repeat units of the polymer may be substituted with one or more water or C₁₋₈ alcohol-solubilising substituents. A water or C₁₋₈ alcohol solubilising substituent as described herein may enhance solubility of the light-emitting polymer as compared to a polymer in which the water or C₁₋₈ alcohol solubilising substituent is not present, e.g. in which the water or C₁₋₈ alcohol solubilising substituent is replaced with H or a non-polar substituent such as an alkyl substituent.

The water or C₁₋₈ alcohol solubilising substituent may consist of a polar group or may comprise one or more polar groups. Polar groups are preferably non-ionic groups capable of forming hydrogen bonds or ionic groups.

Each repeat unit of the polymer may be unsubstituted or substituted with one or more substituents. Substituents may be selected from non-polar substituents, for example C₁₋₃₀ hydrocarbyl substituents; and polar substituents. Polar substituents may be ionic or non-ionic.

In the case where the light-emitting group is pendant from a conjugated polymer backbone, the polymer may comprise a host repeat unit substituted with a light-emitting group. It will be understood that only some host repeat units may be substituted with a light-emitting group, e.g. up to 30 mol %, up to 20 mol %, up to 10 mol % or up to 5 mol % of host repeat units.

Optionally, at least one repeat unity of the light-emitting polymer is substituted with a non-ionic group. The non-ionic group may or may not be polar.

In some embodiments, the non-ionic group may be C₁₋₂₀ alkylene or phenylene-C₁₋₂₀ alkylene wherein one or more non-adjacent C atoms may be replace with O, S, N or C═O. In some embodiments, the non-ionic group may be a C₆₋₂₀ arylene or 5-20 membered heteroarylene, more preferably phenylene, which, other than the one or more substituents R¹, may be unsubstituted or substituted with one or more non-polar substituents, optionally one or more C₁₋₂₀ alkyl groups. “Alkylene” as used herein means a branched or linear divalent alkyl chain.

Optionally, at least one repeat unit of the light-emitting polymer is substituted with at least one polar substituent. The polar substituted may be non-ionic.

C₁₋₃₀ hydrocarbyl groups as described anywhere herein include, without limitation, C₁₋₂₀ alkyl, unsubstituted phenyl and phenyl substituted with one or more C₁₋₂₀ alkyl groups.

As used herein “non-ionic polar groups” may refer to one or more groups which render the light-emitting polymer with a solubility of at least 0.0005 mg/ml in an alcoholic solvent, preferably at least 0.001, 0.01, 0.1, 1, 5 or 10 mg/ml. The solubility is measured at 25° C. Preferably, the alcoholic solvent is a C₁₋₁₀ alcohol, more preferably methanol.

In some embodiments, one or more repeat units of the light-emitting polymer may be substituted with non-ionic polar groups of formula —O(R³O)_{q}—R⁴ wherein R³ in each occurrence is a C₁₋₁₀ alkylene group, optionally a C₁₋₅ alkylene group, wherein one or more non-adjacent, non-terminal C atoms of the alkylene group may be replaced with O, R⁴ is H or C₁₋₅ alkyl, and q is at least 1, optionally 1-10. Preferably, q is at least 2. More preferably, q is 2 to 5. The value of q may be the same in all the polar groups of formula —O(R³O)_{q}—R⁴. The value of q may differ between non-ionic polar groups of the same polymer.

By “C₁₋₅ alkylene group” as used herein with respect to R³ is meant a group of formula —(CH₂)_{f}— wherein f is from 1-5.

Preferably, the polymer comprises non-ionic polar groups of formula —O(CH₂CH₂O)_{q}R⁴ wherein q is at least 1, optionally 1-10 and R⁴ is a C₁₋₅ alkyl group, preferably methyl. Preferably, q is at least 2. More preferably, q is 2 to 5, most preferably q is 3.

In some embodiments, at least one repeat unit is substituted with non-ionic polar groups of formula —N(R⁵)₂, wherein R⁵ is H or C₁₋₁₂ hydrocarbyl. Preferably, each R⁵ is a C₁₋₁₂ hydrocarbyl.

The host repeat unit is substituted with a polar group. The polymer may comprise one or more polar groups. In some embodiments, at least about 90%, 91%, 92%, 93%, 94%, 95%, 96%, 97%, 98%, 99% of the host repeat units are substituted with the polar group. In a preferred embodiment, each (e.g. 100%) host repeat unit is substituted with the polar group. The polar group may be an ionic group (e.g. the host repeat unit is substituted with one or more ionic groups). Each of the host repeats unit may be substituted with an ionic group. An ionic group as described herein may be anionic or cationic.

Exemplary anionic groups are —COO⁻, a sulfonate group; hydroxide; sulfate; phosphate; phosphinate; or phosphonate. The counter cation of an anionic group may be selected from a metal cation, optionally Li⁺, Na⁺, K⁺, Cs⁺, preferably Cs⁺, and an organic cation, optionally ammonium, such as tetraalkylammonium, ethylmethyl imidazolium or pyridinium.

An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl. The counter anion of a cationic group may be a halide; a sulfonate group, optionally mesylate or tosylate; hydroxide; carboxylate; sulfate; phosphate; phosphinate; phosphonate; or borate.

In some embodiments, the polymer comprises ionic groups and non-ionic polar groups selected from groups of formula —O(R³O)_{q}—R⁴, groups of formula —N(R⁵)₂ and groups of formula OR⁴. Preferably, the polymer comprises ionic groups of formula —COO⁻ and non-ionic polar groups selected from groups of formula —O(CH₂CH₂O)_{q}R⁴ and groups of formula —N(R⁵)₂.

A polar substituent may have formula -Sp-(R¹)ₙ wherein Sp is a spacer group; R¹ is a polar group as described above; and n is at least 1, optionally 1, 2, 3 or 4.

Preferably, Sp is selected from:
C₁₋₂₀ alkylene or phenylene-C₁₋₂₀ alkylene wherein one or more non-adjacent C atoms may be replace with O, S, N or C═O;
a C₆₋₂₀ arylene or 5-20 membered heteroarylene, more preferably phenylene, which, other than the one or more substituents R¹, may be unsubstituted or substituted with one or more non-polar substituents, optionally one or more C₁₋₂₀ alkyl groups.

Exemplary host arylene repeat units are selected from formulae (III)-(VI):

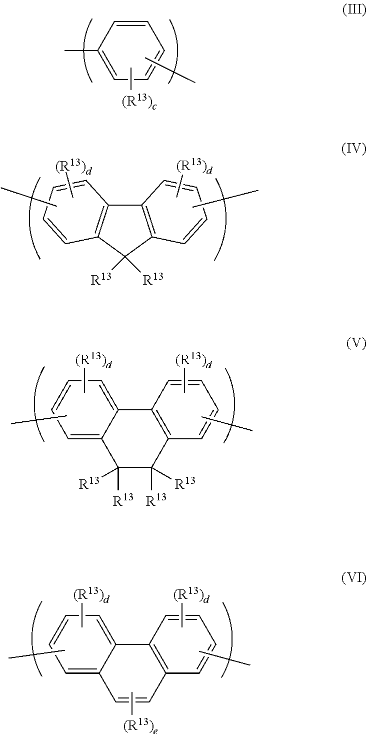

wherein R¹³ in each occurrence is independently a substituent; c is 0, 1, 2, 3 or 4, preferably 1 or 2; each d is independently 0, 1, 2 or 3, preferably 0 or 1; and e is 0, 1 or 2, preferably 2.

Exemplary host repeat units in the backbone of the polymer are:

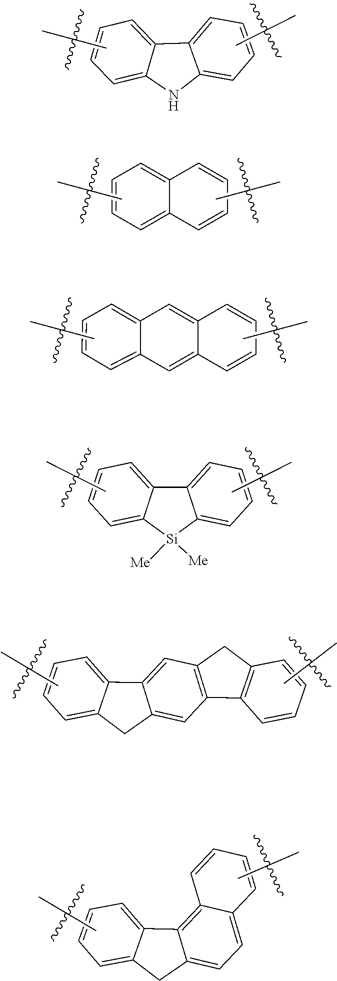

In some preferred embodiments, at least one R¹³ is an ionic substituent comprising or consisting of an ionic group as described herein.

Optionally, an ionic group R¹³ has formula -(Sp)ᵤ-(R²)ᵥ wherein Sp is a spacer group as described above; u is 0 or 1; R² in each occurrence is an ionic group; v is 1 if u is 0; and v is at least 1, optionally 1, 2 or 3, if u is 1.

The backbone of the light-emitting polymer may comprise repeat units of formula (I):

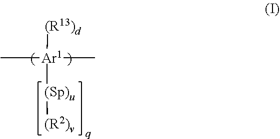

wherein Ar¹ is an arylene group; Sp is a spacer group as previously described; u is 0 or 1; R² is a polar group, preferably an ionic group; v is 1 if u is 0 and v is at least 1 if u is 1; q is at least 1; and R¹³ is a non-ionic group which may or may not be polar.

A preferred host arylene repeat unit has formula (IVa):

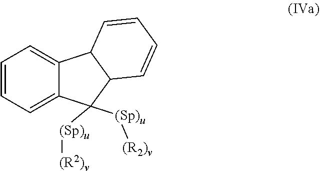

An exemplary repeat unit of formula (IVa) is:

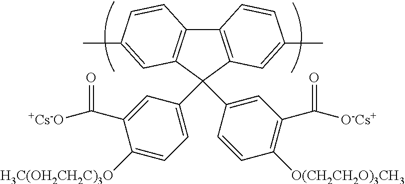

Light-emitting repeat units comprising or consisting of one or more unsubstituted or substituted 5-20 membered heteroarylene groups in the polymer backbone include, without limitation, thiophene repeat units, bithiophene repeat units, benzothiadiazole repeat units, and combinations thereof. Exemplary heteroarylene light-emitting repeat units include repeat units of formulae (VIII), (VIIIa), (IX) and (IXa):

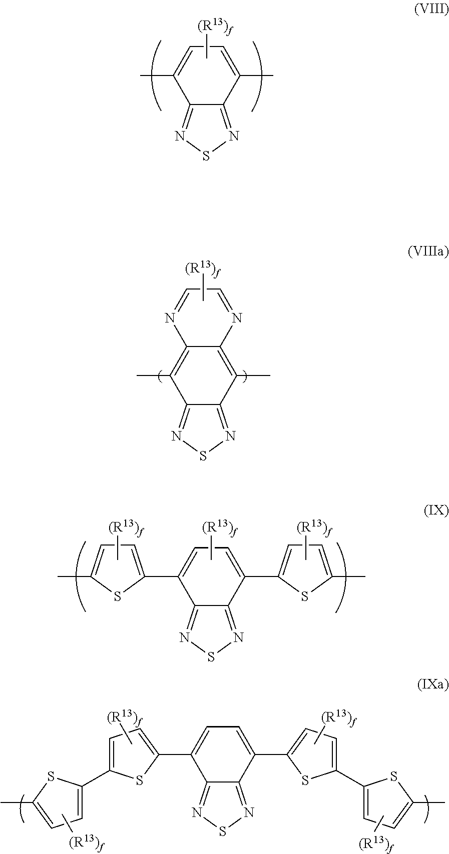

wherein R¹³ in each occurrence is independently a substituent and f is 0, 1 or 2. Each R¹³ may independently be selected from polar and non-polar substituents as described above.

Arylamine light-emitting repeat units may have formula (XII):

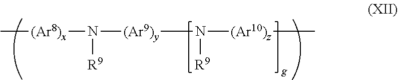

wherein Ar⁸, Ar⁹ and Ar¹⁰ in each occurrence are independently selected from substituted or unsubstituted aryl or heteroaryl, g is 0, 1 or 2, preferably 0 or 1, R¹³ independently in each occurrence is a substituent, and x, y and z are each independently 1, 2 or 3.

R⁹, which may be the same or different in each occurrence when g is 1 or 2, is preferably selected from the group consisting of alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from A⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl, which may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, which may be unsubstituted or substituted with one or more substituents. It is particularly preferred that Ar⁹ is anthracene when g=1. An exemplary group Ar⁹ includes the following, which may be unsubstituted or substituted with one or more substituents, and where * represents a point of attachment to N:

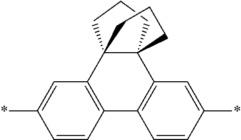 [SMILES: *c1ccc2c(c1)[C@]13CCC[C@]1(CCC3)c1cc(*)ccc1-2]

R⁹ is preferably Ar¹¹ or a branched or linear chain of Ar¹¹ groups, Ar¹¹ in each occurrence is preferably phenyl that may be unsubstituted or substituted with one or more substituents.

Exemplary groups R⁹ include the following, each of which may be unsubstituted or substituted with one or more substituents, and wherein * represents a point of attachment to N:

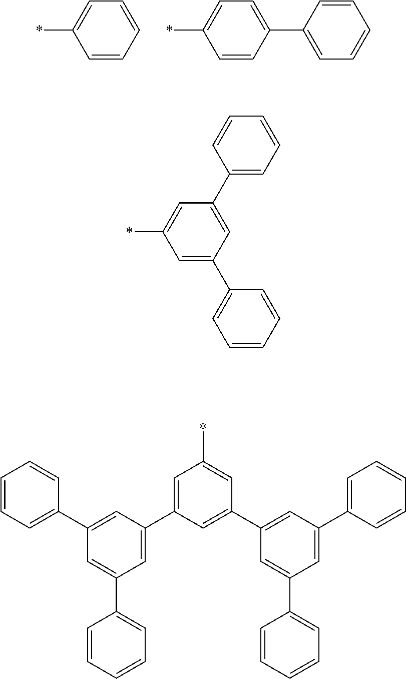

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be selected from non-polar or polar substituents as described herein.

Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (XII) include unsubstituted or substituted units of formulae (XII-1), (XII-2) and (XII-3):

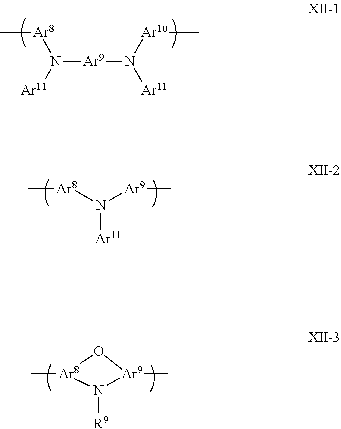

The repeat units may be of formula (XIII), where Q and Z are each independently an aromatic group, and Y is oxygen, sulfur, NH or NR⁹:

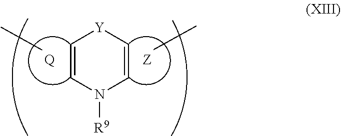

Preferably, each of Q and Z is independently a C₆₋₂₀ aryl, more preferably phenyl or a polycyclic aromatic group.

Exemplary light-emitting repeat units in the backbone of a polymer are:

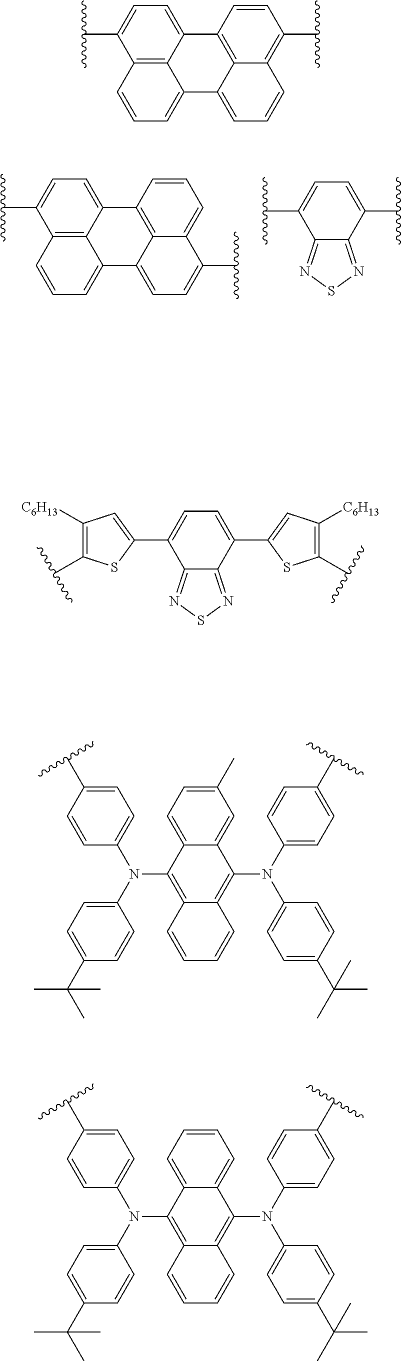

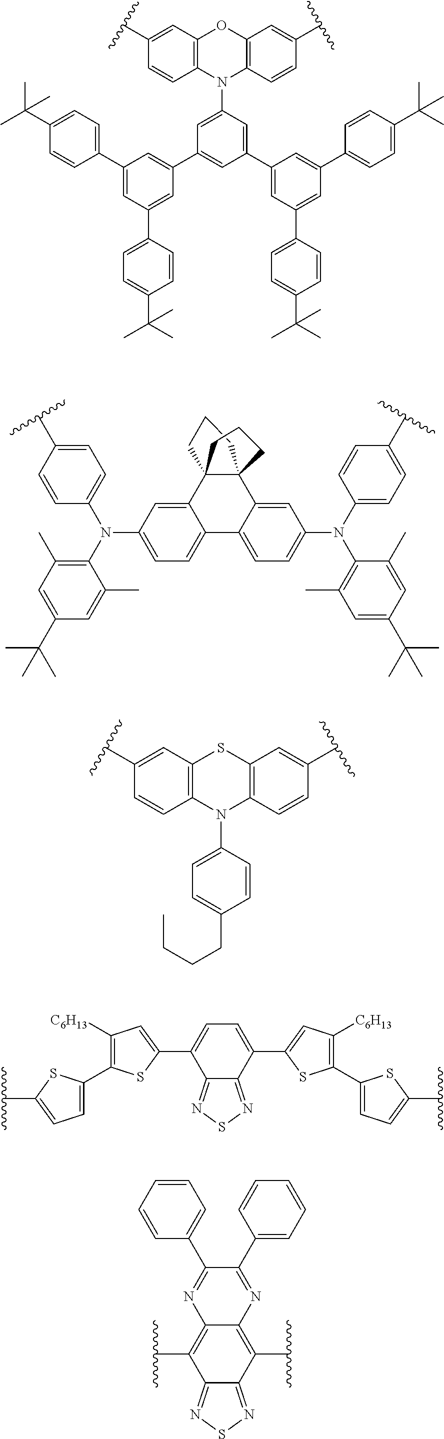

If the light-emitting group is pendant from the backbone of a light-emitting polymer, e.g. a conjugated or non-conjugated light-emitting polymer, then the light-emitting group may be selected from arylene, heteroarylene and arylamine repeat units as described herein wherein the light-emitting group is bound to the polymer backbone through one of the bonds shown for the repeat units, the other bond of the repeat unit being bound to H or a substituent, optionally a C₁₋₃₀ hydrocarbyl group as described herein.

The light-emitting group may be bound directly to the polymer backbone or bound thereto by a divalent binding group, optionally a divalent binding group selected from phenylene which is unsubstituted or substituted with one or more C₁₋₁₂ alkyl groups; and C₁₋₂₀ alkylene wherein one or more non-adjacent C atoms may be replaced with O, S, COO, CO or phenylene.

Exemplary light-emitting repeat units comprising a pendant light-emitting group are:

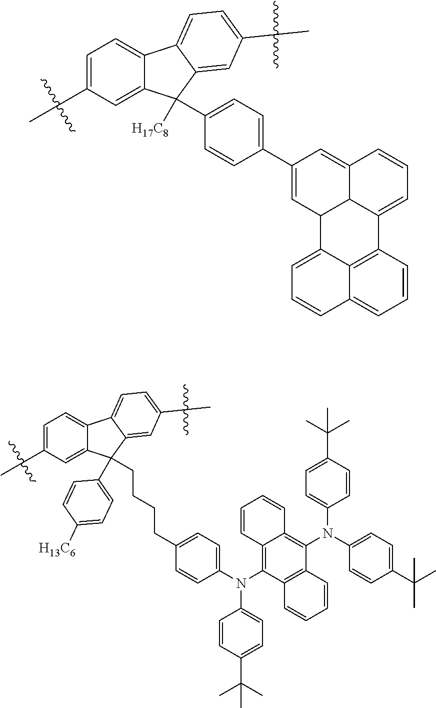

In the case of a phosphorescent conjugated polymer a phosphorescent group, preferably a metal complex, more preferably an iridium complex, may be provided in the main chain, in a side group and/or as an end group of the polymer. An exemplary phosphorescent group is:

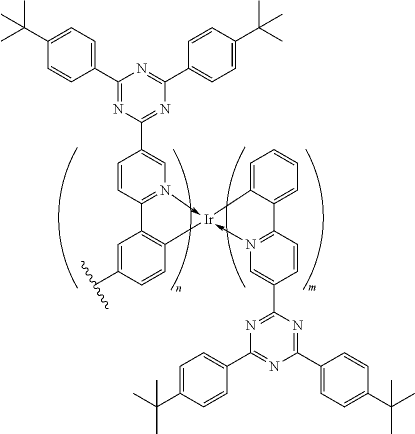

wherein n is 1 and u is 2, or n is 2 and u is 1.

If n is 1 then the light-emitting group may be provided as an end-group of a light-emitting polymer or a side group pendant from a backbone of a light-emitting polymer.

If n is 2 then the light-emitting group may be a light-emitting repeat unit in the backbone of a light-emitting polymer.

Other exemplary phosphorescent groups are:

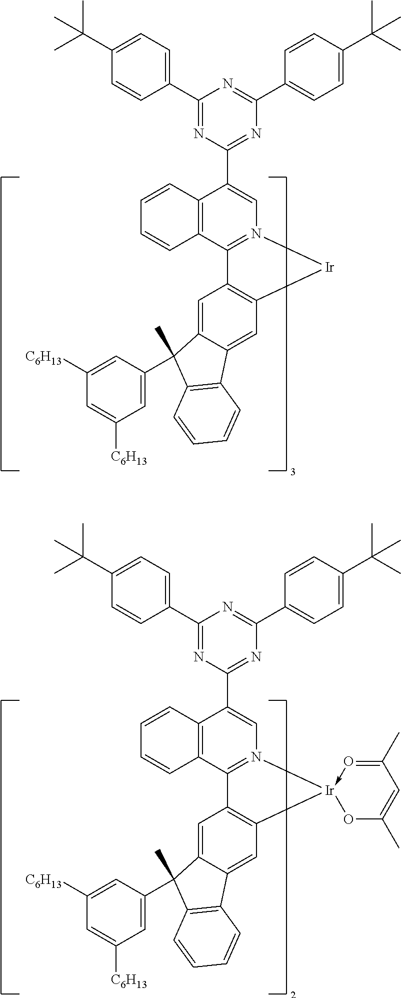

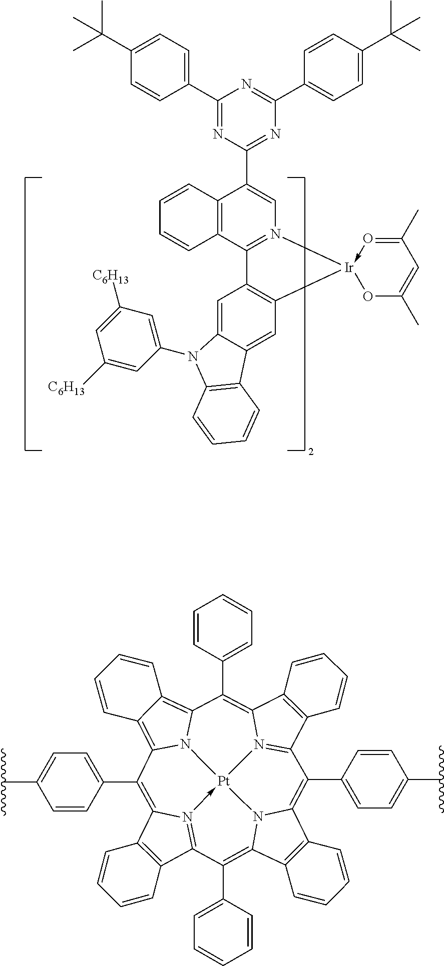

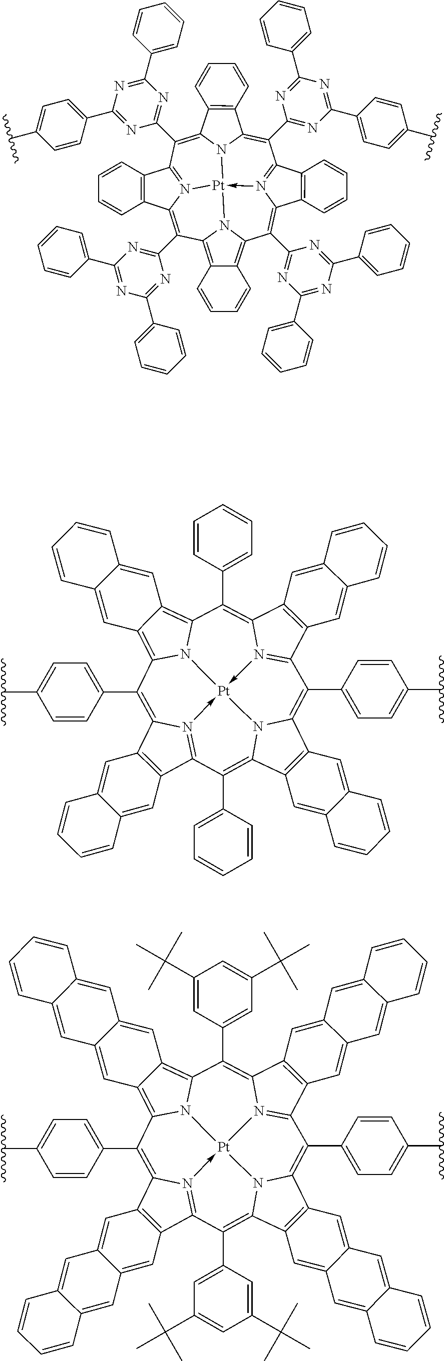

The above iridium and platinum phosphorescent groups may emit light with a peak in the near-infrared region of the photoluminescence spectrum. For example, they may emit light with a peak of between 650 nm up to 1200 nm, optionally 700 nm up to 1200 nm.

The above iridium and platinum emitters may emit light with a peak at a wavelength of greater than about 700 nm, about 720 nm, about 740 nm, about 760 nm, about 780 nm, about 800 nm, about 820 nm, about 840 nm, about 860 nm, about 880 nm, about 900 nm, about 920 nm, about 940 nm, about 960 nm, about 980 nm, about 1000 nm, about 1020 nm, about 1040 nm or about 1060 nm.

The polystyrene-equivalent number-average molecular weight (Mn) measured by gel permeation chromatography of a light-emitting polymers described herein may be in the range of about 1×10³ to 1×10⁸, and preferably 1×10⁴ to 5×10⁶. The polystyrene-equivalent weight-average molecular weight (Mw) of the light-emitting polymers described herein may be 1×10³ to 1×10⁸, and preferably 1×10⁴ to 1×10⁷.

### Colloids

The particles may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid.

The liquid may be a solution comprising salts dissolved therein, optionally a buffer solution.

### Applications

The particles of the present disclosure may be fluorescent or phosphorescent. Preferably the particles are fluorescent. Preferably the particles are for use as a fluorescent probe for detecting a biomolecule or for labelling a biomolecule. In some embodiments, the particles may be used as a fluorescent probe in an immunoassay such as a lateral flow or solid state immunoassay. Optionally the particles are for use in fluorescence microscopy, flow cytometry, next generation sequencing, in-vivo imaging, or any other application where light-emitting polymer(s) is/are brought into contact with a sample to be analysed. The applications can be for medical, veterinary, or environmental applications whether involving patients (where applicable) or for research purposes.

Particles as described herein may be used in a multiplexed assay in which different particles having light-emitting polymers emitting different wavelengths are used, Preferably, the different particles can be caused to emit by illumination from a single light source.

In use the biomolecule binding group of the particles may bind to target biomolecules which include without limitation DNA, RNA, peptides, carbohydrates, antibodies, antigens, enzymes, proteins and hormones. It will be understood that the biomolecule binding group may be selected according to the target biomolecule.

A sample to be analysed may brought into contact with the particles, for example the particles in a colloidal suspension. The target biomolecule may be immobilised on a surface which is brought into contact with the particles.

In some embodiments, the particles may be stored in a dry, optionally lyophilised, form.

### Example 1

Light Emitting Polymer Example 1 (LEP1), which is a green light-emitting polymer, was formed by Suzuki polymerisation of 97 mol % of monomers for forming Host Repeat Unit 1 (50 mol % diboronic ester monomers and 47 mol % dibromo monomers) and 3 mol % of a dibromo monomer for forming Light-Emitting Repeat Unit 1 (which is a mixture of isomers). It will be understood that LEP1 formed by this process contains chains of fluorene repeat units interrupted at random points along the chain by the light-emitting repeat units. It will be understood that no light-emitting repeat units formed by this process are adjacent.

Nanoparticles were formed by polymerisation of tetraethylorthosilicate in a solution containing LEP1 by the process described in WO 2018/060722, the contents of which are incorporated herein by reference.

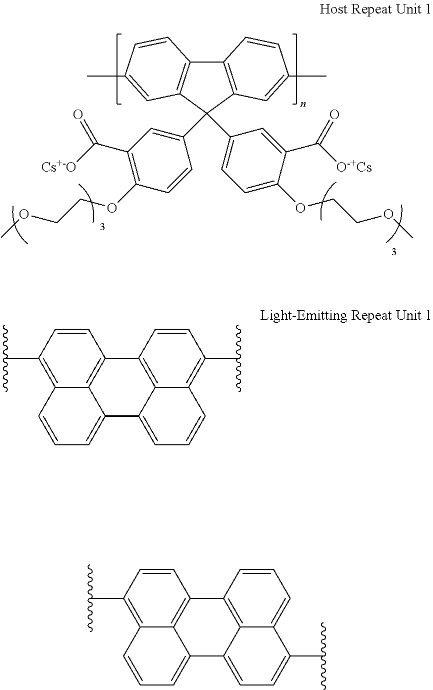

### Example 2

Nanoparticles were formed as described for Example 1 except that Light-Emitting Polymer Example 2 (LEP2), which is a red light-emitting polymer, was used in place of LEP1.

LEP2 was formed as described for LEN except that LEP2 was formed using a dibromo-monomer for forming Light-Emitting Repeat Unit 2 in place of the monomer for forming Light-Emitting Repeat Unit 1:

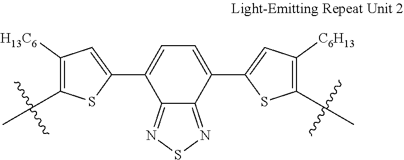

### Comparative Example 1

Nanoparticles were formed as described for Example 1 except that Comparative Light-Emitting Polymer 1 (Comparative LEP1), which is a green light-emitting polymer, was used in place of LEP1.

Comparative LEP1 was formed by polymerising 70 mol % of monomers for forming Host Repeat Unit 1 (50 mol % diboronic ester monomers and 20 mol % dibromo monomers) and 30 mol % of a monomer for forming Light-Emitting Repeat Unit 3:

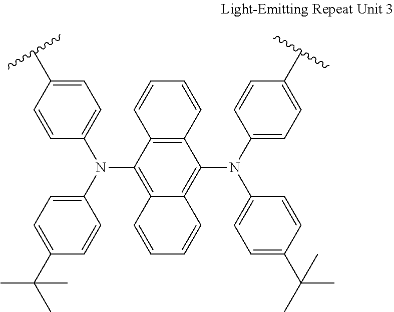

### Solubility of Polymers

Solubility may be measured by weighing the solid polymer into a glass vial. The required amount of polar solvent (for example methanol) is then added followed by a small magnetic stirrer. Next, the vial is tightly capped and put on a preheated hot plate at 60° C. with stirring for 30 min. The polymer solution is then allowed to cool to room temperature before use. The polymer solution can also be prepared by sonicating the polymer containing vial for 30 min at room temperature. The solubility of polymer is tested by visual observation and under white and 365 nm UV light.

### Results

Emission and absorption spectra were measured for each light-emitting polymer in the following forms:
in solution;
in a film formed by spin-coating a solution of the polymer; and
in the nanoparticles described above.

UV/vis spectra in methanol were recorded using a Cary 5000 UV-vis-IR spectrometer. Photoluminescence spectra in the same dispersant were recorded using an Olympus BX62 microscope, with a mercury short arc lamp (λₑₓ=365 nm) for excitation and an Ocean Optics 2000+ spectrometer as the detector.

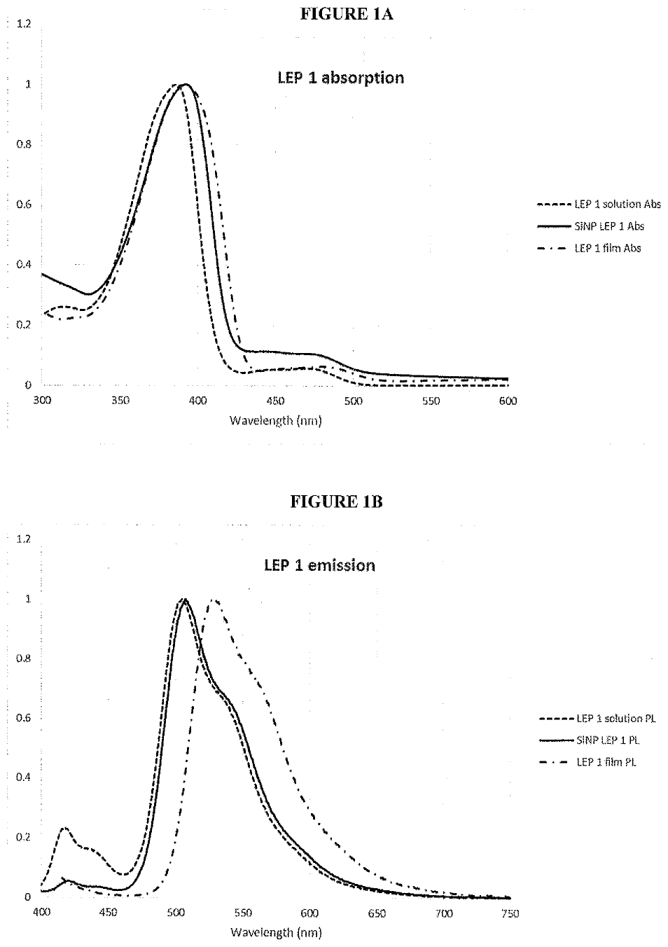

FIGS. 1A and 1B show, respectively, absorption and emission spectra for LEP1, absorption spectra for LEP1 in solution, in a film and in a nanoparticle are similar. Emission is observed from Light-Emitting Repeat Unit 1 at around 500-550 nm. The small proportion (3 mol %) of this repeat unit in the polymer means that emission at around 400 nm is also observed from the fluorene host repeat units when the polymer is in solution. However, the proportion of emission from Light-Emitting Repeat Unit 1 in the nanoparticle is much greater, and close to that observed in the neat film of LEP1. Without wishing to be bound by any theory, this may be due to polymer chains being held in close proximity to one another in the nanoparticle, allowing for efficient energy transfer from the host repeat units to Light-Emitting Repeat Unit 1.

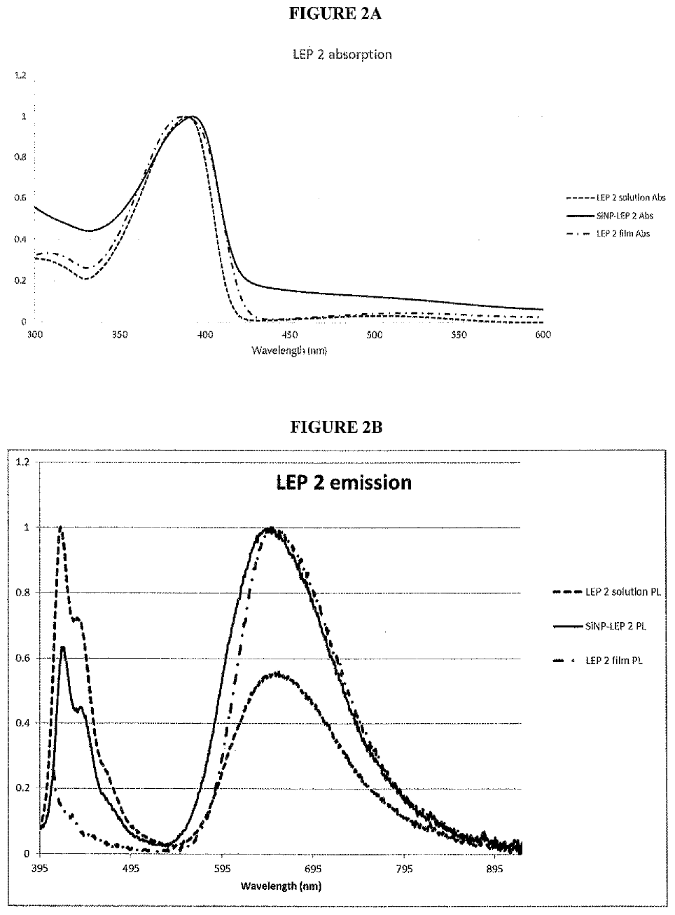

FIGS. 2A and 2B show absorption and emission spectra, respectively, for LEP2 in solution, in a film and in a nanoparticle. Emission is observed from Light-Emitting Repeat Unit 2 at around 650 nm. The small proportion of this repeat unit in the polymer means that substantial emission is also observed from the fluorene host repeat units when the polymer is in solution. However, the proportion of emission from Light-Emitting Repeat Unit 1 in the nanoparticle is much greater, and close to that observed in the neat film of LEP2.

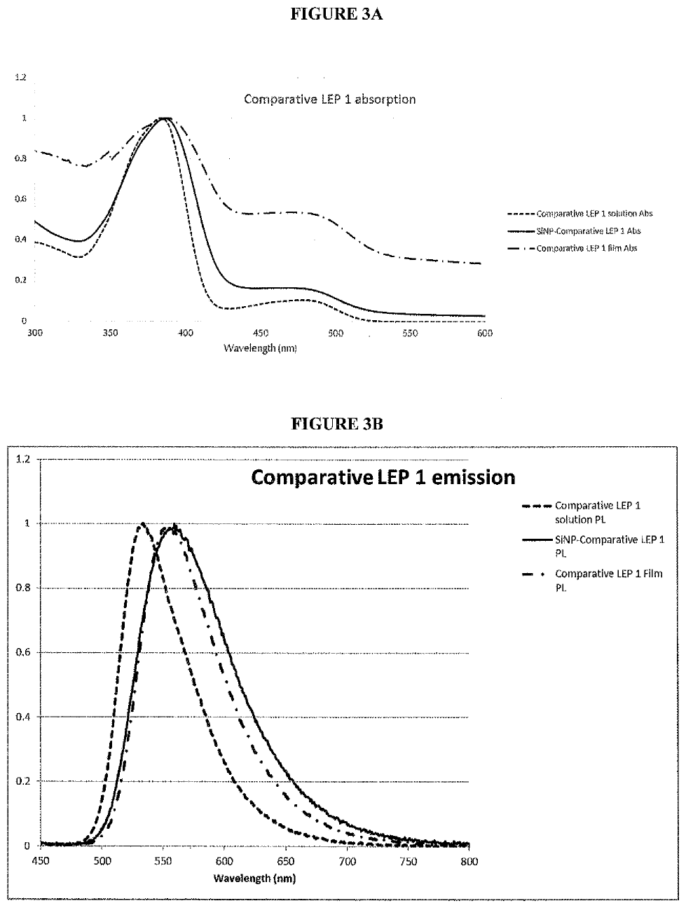

With reference to FIGS. 3A and 3B, which show absorption and emission spectra respectively for Comparative LEP1, an absorption peak appears at around 400-500 nm for the film which is not present in solution. Without wishing to be bound by any theory, this absorption peak is due to the relatively large concentration of light-emitting repeat units in this polymer. This limits the ability of Comparative LEP1 to efficiently absorb excitation energy from a single illumination source at around 380-400 nm.

1. A light-emitting particle comprising an inorganic matrix material and a light-emitting polymer wherein the light-emitting polymer comprises a light-emitting group and a host repeat unit wherein a bandgap of the host repeat unit is greater than that of the light-emitting group and wherein the light-emitting group makes up no more than 10 mol % of the light-emitting polymer.

2. The light-emitting particle according to claim 1 wherein the polymer comprises a light-emitting repeat unit comprising the light-emitting group.

3. The light-emitting particle according to claim 1 wherein the light-emitting polymer is a conjugated polymer.

4. The light-emitting particle according to claim 3 wherein the host repeat unit is an arylene repeat unit which may be unsubstituted or substituted with one or more substituents and which comprises no more than 3 fused aromatic rings.

5. The light-emitting particle according to claim 3 wherein the light-emitting group is a light-emitting repeat unit in a backbone of the polymer and wherein the light-emitting group is unsubstituted or substituted with one or more substituents.

6. The light-emitting particle according to claim 5 wherein the light-emitting repeat unit is selected from heteroarylene repeat units and arylamine repeat units which may be unsubstituted or substituted with one or more substituents.

7. The light-emitting particle according to claim 6 wherein the host repeat unit is a fluorene repeat unit which may be unsubstituted or substituted with one or more substituents.

8. The light-emitting particle according to claim 1 wherein the light-emitting group is a substituent of a repeat unit of the light-emitting polymer.

9. The light-emitting particle according to claim 1 wherein the light-emitting repeat group makes up no more than 5 mol % of the polymer.

10. The light-emitting particle according to claim 1 wherein an absorption peak wavelength of an absorption spectrum of the light-emitting polymer is at least 100 nm shorter than an emission peak wavelength of an emission spectrum of the light-emitting polymer.

11. The light-emitting particle according to claim 1 wherein the particle comprises a biomolecule binding group configured to bind to a target biomolecule.

12. The light-emitting particle according to claim 1 wherein the inorganic matrix comprises silica.

13. A colloidal suspension comprising light-emitting particles according to claim 1 suspended in a liquid.

14. A colloidal suspension according to claim 13 wherein the liquid is a protic liquid.

15. (canceled)

16. A process for preparing a light-emitting particle according to claim 12, comprising formation of the silica by polymerisation of a silica monomer in the presence of the light-emitting polymer.

17. A method of marking a biomolecule, the method comprising the step of binding the biomolecule to a light-emitting particle according to claim 1.

18. A light-emitting polymer comprising a light-emitting group and a host repeat unit, wherein a bandgap of the host repeat unit is greater than that of the light-emitting group, wherein the light-emitting group makes up no more than 10 mol % of the light-emitting polymer and wherein the polymer has a solubility in water or a C₁₋₈ alcohol at 20° C. of at least 0.1 mg/mL.

19. A light-emitting polymer comprising a light-emitting group and a host repeat unit substituted with an ionic group, wherein a bandgap of the host repeat unit is greater than that of a light-emitting group and wherein the light-emitting group makes up no more than 10 mol % of the light-emitting polymer.

20-33. (canceled)

34. A solution comprising the light-emitting polymer according to claim 18.

35-36. (canceled)
